(19) United States
(12) Patent Application Publication
(10) Pub. No.: US 2026/0237065 A1
(43) Pub. Date: Aug. 13, 2026

(54) METHOD AND SYSTEM FOR DETERMINING A DEFORMATION OF A BLOOD VESSEL

(71) Applicant: ViTAA Medical Solutions Inc., Montreal (CA)

(72) Inventors: Richard BEDDOES, Montreal (CA); Elena DI MARTINO, Calgary (CA); Arianna FORNERIS, Calgary (CA); Harold WODLINGER, Toronto (CA)

(21) Appl. No.: 19/156,973

(22) PCT Filed: Feb. 16, 2024
(86) PCT No.: PCT/IB2024/051521

§ 371 Date: Aug. 15, 2025

Related U.S. Application Data
(60) Provisional application No. 63/485,560, filed on Feb. 17, 2023.

(51) Int. Cl. G06T 7/00; G06T 3/147; G06T 3/153; G06T 7/33; G06T 7/62

(52) U.S. Cl. CPC G06T 7/0012; G06T 3/147; G06T 3/153; G06T 7/344; G06T 7/62; G06T 2207/30101; G06T 2207/30172

(57) ABSTRACT

There is described a method for determining a deformation in a blood vessel of a subject, the method being executed by a processor, the method comprising: receiving an initial 3D model of the blood vessel of the subject, the initial 3D model having been generated from an initial medical image of the blood vessel acquired at an initial point in time; receiving a subsequent 3D model of the blood vessel of the subject, the subsequent 3D model having been generated from a subsequent medical image of the blood vessel acquired at a subsequent point in time; registering the initial 3D model to the subsequent 3D model, thereby obtaining registered 3D models; and determining at least one deformation parameter of the blood vessel based on the registered 3D models.

### CROSS-REFERENCE TO RELATED APPLICATIONS

The present application is a 371 application of international PCT Application PCT/IB2024/051521, filed 16 Feb. 2024; which claims the benefit of and priority to and U.S. Provisional Application Ser. No. 63/485,560 filed on Feb. 17, 2023.

### FIELD

The present technology pertains to the field of medical imaging. More specifically, the present technology relates to methods and systems for determining a deformation of a blood vessel.

### BACKGROUND

Measuring the geometric change in blood vessels is a critical component in understanding the progression and changes that the vessel undergoes over time. These changes can reflect underlying pathophysiological conditions that are unique to each pathological vessel and can be a strong indication of impending rupture in aortic aneurysms. The rate of expansion in vessels has also been shown to be complex and understanding deformations in 3-dimensions (3D) is important in capturing the nuances of vessel growth in monitoring abdominal aortic aneurysms (AAA). Controversy exists in the precision of the current standards in radiological care with respect to measurements of the aneurysmal sac size. Confidence in manual measurements is estimated to be between 3 mm and 4 mm, or +/−10% of the size of a normal aorta. Along with human error introduced to the measurement of aneurysmal sac size are the geometric deformation artifacts that can occur due to accuracy issues in different imaging modalities such as computed tomography (CT), magnetic resonance (MR), ultrasound (US), or the like. Errors can also be introduced into the growth analysis through changes in acquisition parameters (resolution), or from the motion of the heart over the cardiac cycle. Generating consistent reproducible measurements of the deformed vessel is critical in understanding the progression of a diseased vessel and eliminating false measurements due to artifacts in the imaging acquisition.

Therefore, there is a need for a method and system for determining a deformation in a blood vessel.

### SUMMARY

It is an object of the present technology to provide an automatic detection of a deformation of a vessel blood such as a growth of a blood vessel.

The present technology minimizes the above-described errors through the use of shape-based distributions to correct for both noise artifacts and bulk geometric disparity. In some embodiments, the purpose of the present technology is to provide a method that can be applied to a 3D image of vasculature geometry to detect the changes in the geometric features of the vessel for aortic wall and lumen diameter changes and deformations. The present technology also provides local measurements of the deformation and evolution of localized change in the blood vessel geometry. A localized change can be measured using consistent landmarks in the geometry available at each time-point, along with deformations relative to the structure of the vessel itself.

In at least some embodiments, two 3D models of a blood vessel are generated from two images or scans captured at different points in time and registration between the two 3D models is used for the assessment of local geometric changes to the blood vessel over time. Those changes can be used to observe the changes in the local structure of the blood vessel while the registration is used to generate a shape-based estimate of the local deformation of the surface, and distances through subsequent scans. The use of shape and probability-based surface deformation estimations to generate correspondence between the points of the 3D models allows the comparison of multi-modal image acquisitions, i.e., the deformation of the blood vessel can be compared between CT, MR, ultrasound, and any modality that produces 3D images.

In at least some embodiments, in order to measure the surface deformation of the blood vessel structure, a deformation gradient is computed from the change in the 3D model of the blood vessel from the initial scan to the subsequent scan.

In at least some embodiments, using landmarks along the blood vessel geometry, such as arterial bifurcations, the relative position of measurements are obtained to align measurements from the initial scan to a subsequent scan. These measurements may include the measurement and distribution of the maximum, minimum, and/or average blood vessel diameters. The change in volume of both the lumen and wall geometry may also be computed based on the volume contained within the given landmarks for both scans.

This present technology allows for automatically determining changes in a blood vessel between two 3D images of the blood vessel taken at different points in time. The changes can be determined without intensity, pixel or voxel information.

By performing the analysis on a 3D model generated based on acquired images, the registration step can be applied to all imaging modalities where reconstruction is created from a segmented structure. As a result, the present technology is modality agnostic, i.e., it can be used between modalities, such as CT, MR, and ultrasound. It can also simplify and provide methods to compare between imaging modalities.

The present technology allows for clinicians to reliably evaluate localized changes in a blood vessel geometry over time. For example, the identification of a high degree of local growth in the neck or landing zones of an aorta and/or iliac arteries may assist a clinician in determining whether an EVAR stent would potentially migrate or develop an endoleak. As another example, the identification of a high degree of local growth in an aortic aneurysm which has a maximum diameter below that usually treated, may indicate that treatment is necessary to avoid an eventual rupture.

According to a first broad aspect, there is provided a method for determining a deformation in a blood vessel of a subject, the method being executed by a processor, the method comprising: receiving an initial 3D model of the blood vessel of the subject, the initial 3D model having been generated from an initial medical image of the blood vessel acquired at an initial point in time; receiving a subsequent 3D model of the blood vessel of the subject, the subsequent 3D model having been generated from a subsequent medical image of the blood vessel acquired at a subsequent point in time; registering the initial 3D model to the subsequent 3D model, thereby obtaining registered 3D models; and determining at least one deformation parameter of the blood vessel based on the registered 3D models.

In one or more embodiments, the step of receiving the subsequent 3D model comprises: receiving the subsequent medical image of the blood vessel; segmenting the subsequent medical image, thereby obtaining a subsequent segmented image; and generating the subsequent 3D model of the blood vessel based on the subsequent segmented image.

In one or more embodiments, the step of receiving the subsequent 3D model comprises: receiving the initial medical image of the blood vessel; segmenting the initial medical image, thereby obtaining an initial segmented image; and generating the initial 3D model of the blood vessel based on the initial segmented image.

In one or more embodiments, the initial 3D model and the subsequent 3D model each comprise a 3D surface model.

In one or more embodiments, the 3D surface model comprises a polygonal model.

In one or more embodiments, the step of registering the initial 3D model to the subsequent 3D model is performed using a rigid registration method and a deformable registration method.

In one or more embodiments, the step of registering the initial 3D model to the subsequent 3D model is performed using a rigid registration method, an affine registration method and a deformable registration method.

In one or more embodiments, the method further comprises identifying landmarks in the 3D initial model and the 3D subsequent model.

In one or more embodiments, the step of registering the initial 3D model to the subsequent 3D model is performed based on the identified landmarks.

In one or more embodiments, the method further comprises rescaling one of the initial 3 D model and the subsequent 3D model based on the identified landmarks.

In one or more embodiments, the step of registering the initial 3D model to the subsequent 3D model comprises: determining a first centerline of the initial 3D model; determining a second centerline of the subsequent 3D model; and registering the first centerline to the second centerline.

In one or more embodiments, the step of determining at least one deformation comprises determining a deformation gradient based on the registered 3D models.

In one or more embodiments, the step of determining at least one deformation comprises determining a given deformation parameter for the initial 3D model and determining the given deformation parameter for the subsequent 3D model.

In one or more embodiments, the given deformation parameter comprises at least one of a diameter, a volume, a length, a surface area, an equivalent circle diameter and an asymmetry.

According to another broad aspect, there is provided a system for determining a deformation in a blood vessel of a subject, the system comprising: a processor; and a non-transitory storage medium operatively connected to the processor, the non-transitory storage medium comprising computer-readable instructions stored thereon; the processor, upon executing the computer-readable instructions, being configured for: receiving an initial 3D model of the blood vessel of the subject, the initial 3D model having been generated from an initial medical image of the blood vessel acquired at an initial point in time; receiving a subsequent 3D model of the blood vessel of the subject, the subsequent 3D model having been generated from a subsequent medical image of the blood vessel acquired at a subsequent point in time; registering the initial 3D model to the subsequent 3D model, thereby obtaining registered 3D models; and determining at least one deformation of the blood vessel based on the registered 3D models.

In one or more embodiments, the processor is further configured for: receiving the subsequent medical image of the blood vessel; segmenting the subsequent medical image, thereby obtaining a subsequent segmented image; and generating the subsequent 3D model of the blood vessel based on the subsequent segmented image.

In one or more embodiments, the processor is further configured for: receiving the initial medical image of the blood vessel; segmenting the initial medical image, thereby obtaining an initial segmented image; and generating the initial 3D model of the blood vessel based on the initial segmented image.

In one or more embodiments, the initial 3D model and the subsequent 3D model each comprise a 3D surface model.

In one or more embodiments, the 3D surface model comprises a polygonal model.

In one or more embodiments, the processor is configured for registering the initial 3D model to the subsequent 3D model using a rigid registration method and a deformable registration method.

In one or more embodiments, the processor is configured for said registering the initial 3D model to the subsequent 3D model using a rigid registration method, an affine registration method and a deformable registration method.

In one or more embodiments, the processor is further configured for identifying landmarks in the 3D initial model and the 3D subsequent model.

In one or more embodiments, the processor is configured for said registering the initial 3D model to the subsequent 3D model based on the identified landmarks.

In one or more embodiments, the processor is further configured for rescaling one of the initial 3 D model and the subsequent 3D model based on the identified landmarks.

In one or more embodiments, the processor is configured for: determining a first centerline of the initial 3D model; determining a second centerline of the subsequent 3D model; and registering the first centerline to the second centerline.

In one or more embodiments, said determining at least one deformation comprises determining a deformation gradient based on the registered 3D models.

In one or more embodiments, the processor is configured for determining a given deformation parameter for the initial 3D model and determining the given deformation parameter for the subsequent 3D model.

In one or more embodiments, the given deformation parameter comprises at least one of a diameter, a volume, a length, a surface area, an equivalent circle diameter and an asymmetry.

### Definitions

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one physical computer or one physical computer system, but neither is required to be the case with respect to the present technology. In the present context, the use of the expression “a server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e., its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that the use of the terms “server” and “third server” is not intended to imply any particular order, type, chronology, hierarchy or ranking (for example) of/between the servers, nor is their use (by itself) intended to imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 depicts a schematic diagram of an electronic device in accordance with one or more non-limiting embodiments of the present technology.

FIG. 2 depicts a schematic diagram of a communication system in accordance with one or more non-limiting embodiments of the present technology.

FIG. 3 depicts a schematic diagram of a vessel deformation detection procedure being executed within the system of FIG. 2 in accordance with one or more non-limiting embodiments of the present technology.

FIG. 4A illustrates an initial 3D model of an aorta and a subsequent 3D model of the aorta during a registration process.

FIG. 4B illustrates the centerlines of the initial and subsequent 3D models of FIG. 4A prior and after the registration procedure.

FIG. 5 illustrates obtaining subsections of data set using a common geometric space defined by the centerline of a vessel.

FIG. 6 shows a distribution of diameter measurements at a single level of a vessel geometry taken at a given point along a centerline thereof.

FIGS. 7A-7C show a distribution of a change in growth measurements generated for a vessel measured at a given set number of divisions that are oriented normal to a centerline of the vessel.

FIG. 8 illustrates the measures of growth applied to multiple to the data-points on the model, connections, polygons, and regions of the model.

FIG. 9 illustrates an exemplary initial 3D model of a blood vessel registered to a subsequent 3D model of the same blood vessel.

FIG. 10 illustrates an exemplary variation of a deformation parameter for a blood vessel.

FIG. 11 illustrates an exemplary variation of a deformation parameter for different views around a blood vessel.

FIG. 12A illustrates exemplary circles positioned along the length of a blood vessel, each circle defining a landmark for defining longitudinal sections.

FIG. 12B illustrates the longitudinal sections of a blood vessel obtained using the circles of FIG. 12A.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some non-limiting embodiments of the present technology, the processor may be a general-purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

With these fundamentals in place, we will now consider some non-limiting examples to illustrate various implementations of aspects of the present technology.

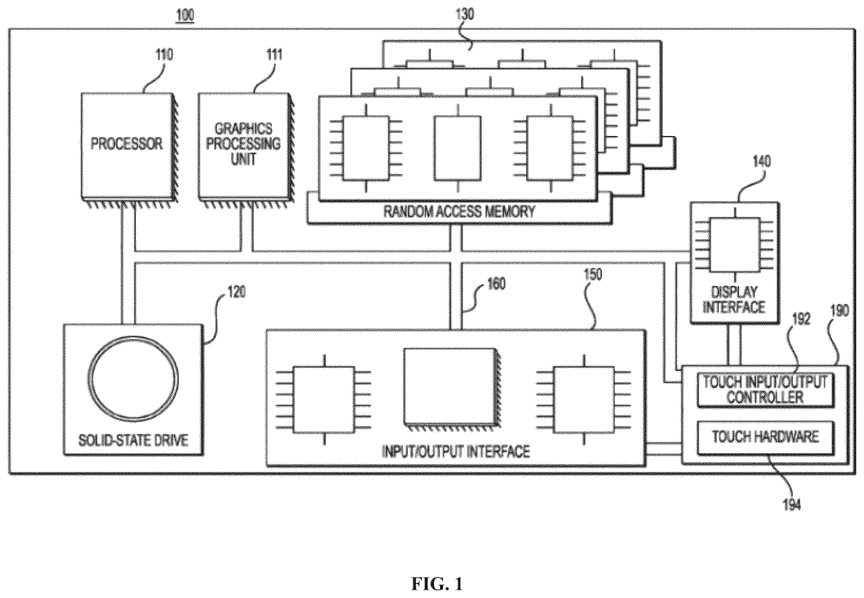

With reference to FIG. 1, there is illustrated a schematic diagram of an electronic device 100 suitable for use with some non-limiting embodiments of the present technology.

### Electronic Device

The electronic device 100 comprises various hardware components including one or more single or multi-core processors collectively represented by processor 110, a graphics processing unit (GPU) 111, a solid-state drive 120, a random-access memory 130, a display interface 140, and an input/output interface 150.

Communication between the various components of the electronic device 100 may be enabled by one or more internal and/or external buses 160 (e.g., a PCI bus, universal serial bus, IEEE 1394 “Firewire” bus, SCSI bus, Serial-ATA bus, etc.), to which the various hardware components are electronically coupled.

The input/output interface 150 may be coupled to a touchscreen 190 and/or to the one or more internal and/or external buses 160. The touchscreen 190 may be part of the display. In some embodiments, the touchscreen 190 is the display. The touchscreen 190 may equally be referred to as a screen 190. In the embodiments illustrated in FIG. 1, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In some embodiments, the input/output interface 150 may be connected to a keyboard (not shown), a mouse (not shown) or a trackpad (not shown) allowing the user to interact with the electronic device 100 in addition or in replacement of the touchscreen 190.

According to implementations of the present technology, the solid-state drive 120 stores program instructions suitable for being loaded into the random-access memory 130 and executed by the processor 110 and/or the GPU 111 for performing in vivo strain mapping of an aortic dissection. For example, the program instructions may be part of a library or an application.

The electronic device 100 may be implemented in the form of a server, a desktop computer, a laptop computer, a tablet, a smartphone, a personal digital assistant or any device that may be configured to implement the present technology, as it may be understood by a person skilled in the art.

### System

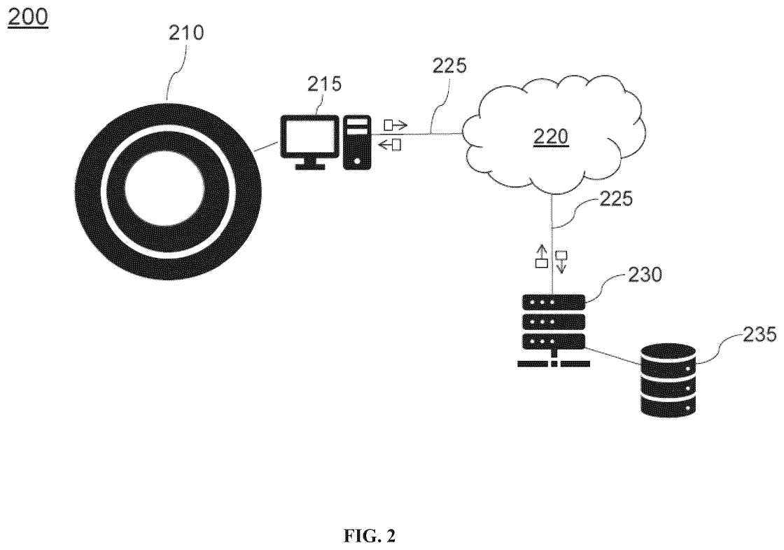

Referring to FIG. 2, there is shown a schematic diagram of a communication system 200, which will be referred to as the system 200, the system 200 being suitable for implementing non-limiting embodiments of the present technology. It is to be expressly understood that the system 200 as illustrated is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 200 may provide in certain instances simple implementations of the present technology, and that where such is the case they have been presented in this manner as an aid to understanding. As persons skilled in the art would understand, various implementations of the present technology may be of a greater complexity.

The system 200 comprises inter alia a medical imaging apparatus 210 associated with a workstation computer 215, and a server 230 coupled over a communications network 220 via respective communication links 225 (not separately numbered).

### Medical Device

The medical imaging apparatus 210 is configured to inter alia acquire, at different time points, a plurality of images of a blood vessel of a given subject such that a 3D representation of the blood vessel of the given subject may be subsequently generated.

In one or more embodiments, the medical imaging apparatus 210 comprises an ECG-gated medical imaging apparatus.

The medical imaging apparatus 210 may comprise one of: a computed tomography (CT) scanner, a magnetic resonance imaging (MRI) scanner, a 3D ultrasound or the like.

In some embodiments of the present technology, the medical imaging apparatus 210 may comprise a plurality of medical imaging apparatuses, such as one or more of a CT scanner, an MRI scanner, a 3D ultrasound scanner, and the like.

The medical imaging apparatus 210 may be configured with specific acquisition parameters for acquiring the plurality of images of a blood vessel during over a cardiac cycle.

As a non-limiting example, in one or more embodiments where the medical imaging apparatus 210 is implemented as a CT scanner, a CT protocol comprising pre-operative retrospectively gated multidetector CT (MDCT-64—row multi-slice CT scanner) with variable dose radiation to capture the R-R interval may be used.

As another non-limiting example, in one or more embodiments where the medical imaging procedure comprises a MRI scanner, the MR protocol can comprise steady state T2 weighted fast field echo (TE=2.6 ms, TR=5.2 ms, flip angle 110 degree, fat suppression (SPIR), echo time 50 ms, maximum 25 heart phases, matrix 256×256, acquisition voxel MPS (measurement, phase and slice encoding directions) 1.56/1.56/3.00 mm and reconstruction voxel MPS 0.78/0.78/1.5), or similar cine acquisition of the portion of aorta under study, axial slices. The medical imaging apparatus 210 includes or is connected to a workstation computer 215 for inter alia data transmission.

### Workstation Computer

The workstation computer 215 is configured to inter alia: (i) control parameters of the medical imaging apparatus 210 and cause acquisition of images; and (ii) receive and process the plurality of images from the medical imaging apparatus 210.

In one or more embodiments. the workstation computer 215 may receive images in raw format and perform a tomographic reconstruction using known algorithms and software.

The implementation of the workstation computer 215 is known in the art. The workstation computer 215 may be implemented as the electronic device 100 or comprise components thereof, such as the processor 110, the graphics processing unit (GPU) 111, the solid-state drive 120, the random-access memory 130, the display interface 140, and the input/output interface 150.

In one or more other embodiments, the workstation computer 215 may be integrated at least in part into the medical imaging apparatus 210.

In one or more embodiments, the workstation computer 215 is configured according to the Digital Imaging and Communications in Medicine (DICOM) standard for communication and management of medical imaging information and related data.

In one or more embodiments, the workstation computer 215 may store the images in a local database (not illustrated).

The workstation computer 215 is connected to a server 230 over the communications network 220 via a respective communication link 225. In one or more embodiments, the workstation computer 215 may transmit the images and/or multiphase stack to the server 230 and the database 235 for storage and processing thereof.

In one or more embodiments, the multiphase stack comprises a plurality of 3D images each taken at a respective and different point in time or phase. At each phase, the 3D image comprises a plurality of voxels each having associated thereto a respective 3D position and a parameter value such as a color value, a grayscale value, an intensity value, or the like.

### Server

The server 230 is configured to inter alia:
(i) receive an initial 3D model of the blood vessel of the subject, the initial 3D model having been generated from an initial medical image of the blood vessel acquired at an initial point in time;
(ii) receive a subsequent 3D model of the blood vessel of the subject, the subsequent 3D model having been generated from a subsequent medical image of the blood vessel acquired at a subsequent point in time, i.e., the subsequent medical image has been acquired at a second point in time which is later than (or which occurred in time after) the first point in time at which the initial medical image was acquired;
(iii) register the initial 3D model to the subsequent 3D model, thereby obtaining a registered initial model; and
(iv) determine at least one deformation of the blood vessel based on the registered initial model and the subsequent 3D model.

As described in greater detail below, the server 230 may also be configured to generate the initial 3D model of the blood vessel and the subsequent 3D model of the blood vessel. In another embodiment, the generation of the 3D models may be performed by a server other than the server 230.

How the server 230 is configured to do so will be explained in more detail herein below.

The server 230 can be implemented as a conventional computer server and may comprise some or all of the components of the electronic device 100 illustrated in FIG. 2. In an example of one or more embodiments of the present technology, the server 230 can be implemented as a Dell™ PowerEdge™ Server running the Microsoft™ Windows Server™ operating system. Needless to say, the server 230 can be implemented in any other suitable hardware and/or software and/or firmware or a combination thereof. In the illustrated non-limiting embodiment of present technology, the server 230 is a single server. In alternative non-limiting embodiments of the present technology, the functionality of the server 230 may be distributed and may be implemented via multiple servers (not illustrated).

The implementation of the server 230 is well known to the person skilled in the art of the present technology. However, briefly speaking, the server 230 comprises a communication interface (not illustrated) structured and configured to communicate with various entities (such as the workstation computer 215, for example and other devices potentially coupled to the network 220) via the communications network 220. The server 230 further comprises at least one computer processor (e.g., a processor 110 or GPU 111 of the electronic device 100) operationally connected with the communication interface and structured and configured to execute various processes to be described herein.

In one or more embodiments, the server 230 may be implemented as the electronic device 100 or comprise components thereof, such as the processor 110, the graphics processing unit (GPU) 111, the solid-state drive 120, the random-access memory 130, the display interface 140, and the input/output interface 150.

### Database

The database 235 is directly connected to the server 230 but, in one or more alternative implementations, the database 235 may be communicatively coupled to the server 230 via the communications network 220 without departing from the teachings of the present technology. Although the database 235 is illustrated schematically herein as a single entity, it will be appreciated that the database 235 may be configured in a distributed manner, for example, the database 235 may have different components, each component being configured for a particular kind of retrieval therefrom or storage therein.

The database 235 may be a structured collection of data, irrespective of its particular structure or the computer hardware on which data is stored, implemented or otherwise rendered available for use. The database 235 may reside on the same hardware as a process that stores or makes use of the information stored in the database 230 such as the server 230, or it may reside on separate hardware, such as on one or more other electronic devices (not shown) directly connected to the server 230 and/or connected to the communications network 220. The database 230 may receive data from the server 230 for storage thereof and may provide stored data to the server 230 for use thereof.

The database 235 is configured to inter alia:
(i) store images having been acquired by the medical imaging apparatus 210;
(ii) store DICOM multiphase stacks;
(iii) store 3D geometrical models of blood vessels;
(iv) store strain maps of dissected blood vessels; and
(v) store interactive models of dissected blood vessels.

### Communication Network

In some embodiments of the present technology, the communications network 220 is the Internet. In alternative non-limiting embodiments, the communication network 220 can be implemented as any suitable local area network (LAN), wide area network (WAN), a private communication network or the like. It should be expressly understood that implementations for the communication network 220 are for illustration purposes only. How a communication link 225 (not separately numbered) between the workstation computer 215 and/or the server 230 and/or another electronic device (not illustrated) and the communications network 220 is implemented will depend inter alia on how each of the medical imaging apparatus 210, the workstation computer 215, and the server 230 is implemented.

The communication network 220 may be used in order to transmit data packets amongst the workstation computer 215, the server 230 and the database 235. For example, the communication network 220 may be used to transmit requests between the workstation computer 215 and the server 230.

In one or more embodiments, the server 230 may be part of a Picture Archiving and Communication System (PACS).

In another embodiment, the server 230 may be omitted. In this case, the workstation computer 215 is in communication with or connected to the database 235, and is configured to inter alia: (i) receive an initial 3D model of the blood vessel of the subject, the initial 3D model having been generated from an initial medical image of the blood vessel acquired at an initial point in time; receive a subsequent 3D model of the blood vessel of the subject, the subsequent 3D model having been generated from a subsequent medical image of the blood vessel acquired at a subsequent point in time; register the initial 3D model to the subsequent 3D model, thereby obtaining a registered initial model; and determining at least one deformation of the blood vessel based on the registered initial model and the subsequent 3D model.

In some embodiments, the workstation computer 215 may also be configured to generate the initial 3D model of the blood vessel and the subsequent 3D model of the blood vessel.

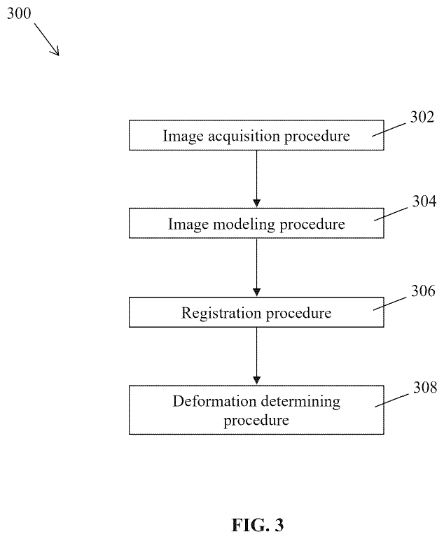

Turning now to FIG. 3, there is illustrated a schematic diagram of a procedure 300 for determining or characterizing a deformation in a blood vessel, in accordance with one or more non-limiting embodiments of the present technology.

The procedure 300 is executed within the system 200 of FIG. 2. In one or more embodiments, the procedure 300 may be executed by the server 230. It is contemplated that some procedures of the AD strain mapping procedure 300 may be executed in parallel by the server 230 or by electronic devices (such as the workstation computer 215) as will be recognized by persons skilled in the art.

The purpose of the procedure 300 is to acquire images of a blood vessel at different points in time, generate a 3D model of the images, register the generated 3D models and determined based on the registered 3D models a deformation in the blood vessel.

The procedure 300 comprises inter alia an image acquisition procedure 302, an image modeling procedure 304, a registration procedure 306 and a deformation determination procedure 308.

### Image Acquisition Procedure

The image acquisition procedure 310 is configured to inter alia:
(i) receive images of a blood vessel or a portion of a blood vessel of a patient having been acquired during a cardiac cycle; and
(ii) generate, using the received images of the dissected blood vessel, a 3D image of the blood vessel or portion of the blood vessel during the cardiac cycle. In one or more embodiments, the blood vessel is an aorta.

In one or more embodiments, the images of the dissected blood vessel are acquired from a subject known to have an aortic aneurysm, which may have been diagnosed by a physician. In one or more other embodiments, the images of the blood vessel may have been acquired without previous knowledge of an aortic aneurysm and may be, for example, detected during the image segmentation procedure 302.

During the image acquisition procedure 310, a plurality of images of a blood vessel, such as an aorta of a given subject, are received. The plurality of images may be received from the workstation computer 215, directly from the medical imaging apparatus 210, from a database such as database 235, etc. In one or more embodiments, the plurality of images of the blood vessel comprises images of an aorta having an aneurysm. It will be appreciated that the type of aortic dissection in the dissected blood vessel is not limited.

In one or more embodiments where the medical imaging apparatus 210 comprises a CT scanner, the CT protocol for CT image acquisition can comprise pre-operative retrospectively gated MDCT (64-row multi-slice CT scanner) with variable dose radiation to capture the R-R interval. In one or more embodiments where the medical imaging apparatus 210 is a MRI scanner, the MR protocol can comprise steady state T2 weighted fast field echo (TE=2.6 ms, TR=5.2 ms, flip angle 110 degree, fat suppression (SPIR), echo time 50 ms, maximum 25 heart phases 2, matrix 256×256, acquisition voxel MPS 1.56/1.56/3.00 mm and reconstruction voxel MPS 0.78/0.78/1.5), or similar cine acquisition of the portion of aorta under study, axial slices.

The image acquisition procedure 310 organizes the plurality of images in a multiphase stack. In one or more embodiments, the plurality of images is organized in phases according to a Digital Imaging and Communications in Medicine (DICOM) stack, the implementation of which is known in the art.

In one or more embodiments, each phase of the multiphase stack corresponds to a time instance in the cardiac cycle of the given patient.

The image acquisition procedure 310 outputs a first or initial 3D image of a blood vessel or blood vessel portion of a subject and a second or subsequent 3D image of the blood vessel or blood vessel portion taken at different points in time. The initial and subsequent 3D images are acquired at the same phase, i.e., at the same instance within the cardiac cycle, but at different points in time. For example, the subsequent 3D image may be acquired weeks or months after the first 3D image. However, the initial 3D image represents the blood vessel of the subject at the reference phase and the subsequent 3D image also represents the blood vessel of the subject at the same reference phase.

### Image Modeling Procedure

The image modeling procedure 304 is configured to inter alia:
(i) receive a 3D image of a blood vessel;
(ii) segment the 3D image; and
(iii) generate a 3D model of the blood vessel based on the segmented 3D image.

It should be understood that any adequate segmentation method for identifying the blood vessel within the received 3D image can be used. For example, the image modeling procedure 304 may use one or more machine learning (ML) models having been trained to recognize blood vessel elements such as the internal surface and the external surface of a blood vessel. In this case, the image modeling procedure 304 may use ML models to perform segmentation by classifying pixels as belonging to a blood vessel surface.

The image segmentation may be performed based on one or more of: pixel intensity, texture, and/or other attributes, using deformable models and techniques such as, but not limited to, low-level segmentation (thresholding, region growing, etc.), model-based segmentation (multispectral, feature maps, dynamic programming, counter following), statistical techniques, fuzzy techniques as well as other techniques known in the art. In one or more other embodiments, at least a portion of the image segmentation may be performed by a human operator by manually drawing the boundaries of the blood vessel.

Once the 3D image has been segmented, the image modeling procedure 304 generates a 3D model of the blood vessel. In some embodiments, the 3D model comprises a 3D surface model, i.e., only the internal and external surfaces of the blood vessel are presented in the 3D model.

In one or more embodiments, the 3D model of the blood vessel comprises a cloud of points. In another embodiment, the 3D models comprise a polygonal mesh. In this case, the image modeling procedure 304 applies a polygon modeling method to obtain the 3D model.

Once generated, the 3D model of the blood vessel is stored in memory.

### Registration Procedure

The registration procedure 306 is configured to inter alia:
(i) receive an initial or first 3D model of the blood vessel and a subsequent 3D model of the blood vessel; and
(ii) register the initial 3D model to the subsequent 3D model.

In one or more embodiments, the registration procedure 306 comprises a scaling step for ensuring that the initial and subsequent 3D models are at the same scale. In one or more embodiments, the initial 3D model is modified, i.e., expanded or scaled down, to be at the same scale as that of the subsequent image. In another embodiments, the subsequent 3D model is modified, i.e., scaled up or scaled down, to be at the same scale as that of the initial 3D model.

In one or more embodiments, landmarks present on the blood vessel (which are represented in the corresponding 3D models) are used for the scaling step, as known in the art.

Once the initial and subsequent 3D models are at the same scale, the registration procedure 306 registers the initial and subsequent 3D models according to the temporal order in which their respective 3D image have been acquires, i.e., it registers the initial 3D model to the subsequent 3D model. It should be understood that any adequate registration method may be used.

In one or more embodiments, by treating the problem in the temporal direction of the change, i.e., by registering the initial 3D model to the subsequent 3D model (but not the subsequent 3D model to the initial 3D model), artifacts arising from false mapping are limited to those related to expansion, rather than erroneous geometric results.

In one or more embodiments, the registration procedure 306 uses the centerline of the 3D model for the registration. In this case, the centerline of the initial 3D model and that of the subsequent 3D model are first determined and the centerline of the initial 3D model is registered to the centerline of the subsequent model.

In one or more embodiments, the registration procedure comprises two registration steps. In this case, a rigid registration is first performed followed by a deformable registration.

In one or more embodiments, the rigid registration is performed using an iterative-closest-point method to provide an alignment of the initial 3D model to the subsequent 3D model so as to reduce or minimize an error metric that measures the goodness of the alignment between the initial and subsequent 3D models. The alignment is performed using a rigid transformation with up to 6 degrees of freedom. During the rigid registration, the initial 3D model may only be rotated and/or translated using a single transformation applied equally to all points of the 3D initial model.

Then, the deformable registration is performed using the rigidly transformed initial 3D model and subsequent 3D model. During the deformable registration, different transforms can be applied to different sections of the rigidly transformed initial 3D model so as to reduce the error metric that is associated with different regions or individual points of the model. The transforms can include rotation(s), translation(s), scaling(s), shearing(s), and/or the like.

In another embodiment, the registration procedure comprises three registration steps: first a rigid registration, then an affine registration and finally a deformable registration.

The rigid registration is first performed to rigidly register the initial 3D model to the subsequent 3D model. The rigidly registered initial 3D model is then registered to the subsequent 3D model using an affine registration. For example, the above-described rigid registration method may be used.

In one or more embodiments, the affine registration uses an iterative-closest-point method to reduce or minimize an error metric measuring the goodness of the alignment between the two models. During the affine registration, the alignment between the models may be performed using an affine transformation, which allows for up to 13 degrees of freedom. During the affine registration, the rigidly registered initial 3D model may only be rotated, translated, scaled and/or sheared using a single transform applied equally to all points of the rigidly registered initial 3D model.

Then the affined registered initial 3D model is further registered to the subsequent 3D model using a deformable registration, as described above.

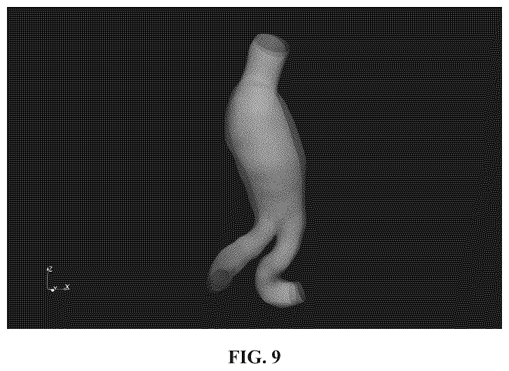

FIG. 4A illustrates an initial 3D model of an aorta and a subsequent 3D model of the aorta during the registration process, i.e., after the rigid registration, after the affine registration and after the deformable registration. FIG. 4B illustrates the centerline of the initial and subsequent 3D models prior and after the registration procedure. As illustrated, the centerlines of the registered initial and subsequent 3D models substantially superimpose on top of each other. FIG. 9 illustrates exemplary initial and subsequent 3D models once the initial 3D model has been registered to the subsequent 3D model.

In a further embodiment, the registration procedure comprises a single registration, i.e., the rigid registration.

The output of the registration procedure 308 comprises the registered initial and subsequent 3D models, i.e., the initial 3D model registered to the subsequent 3D model, which are stored into memory.

### Deformation Determination Procedure

The deformation determination procedure 308 is configured to inter alia:
(i) receive the initial 3D model registered to the subsequent 3D model and the subsequent 3D model; and
(ii) determine, based on the two registered 3D models, a deformation of a blood vessel such a growth of the blood vessel.

In at least some embodiments, the step of determining a deformation of the blood vessel comprises determining at least one deformation parameter for the blood vessel.

A deformation parameter may be one of the following: a volume, a diameter, a surface area, a length, a diameter or equivalent circle diameter, an asymmetry, etc.

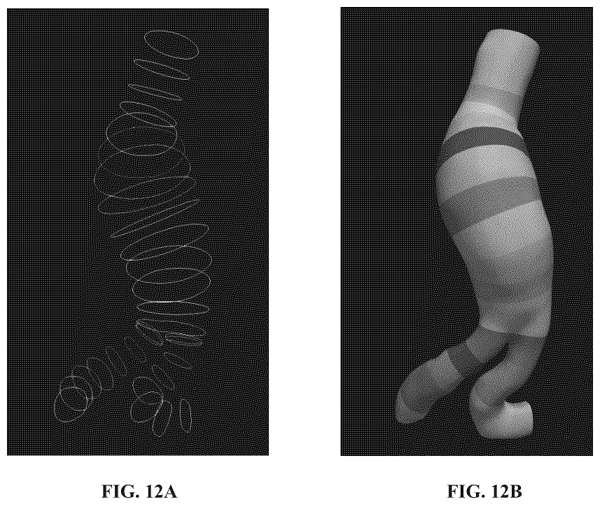

In some embodiments, a section of a blood vessel refers to a longitudinal section of the blood vessel extending along a given portion of the length of the blood vessel, e.g., along a given portion of the centerline of the vessel. A blood vessel may be longitudinally divided into a plurality of sections each extending along a respective length along the blood vessel, and each located at a respective position along the length of the blood vessel. Landmarks positioned along the length of the blood vessel can be used for delimiting/defining the longitudinal sections. The landmarks may be chosen manually by a user or be predefined. FIG. 12A illustrates landmarks in the shape of circle position at different locations along the length of a blood vessel model. The landmarks define the frontier between two longitudinal sections. FIG. 12B illustrate the different longitudinal sections obtained using the landmarks of FIG. 12A.

The volume may correspond to the volume of the lumen of the vessel, the volume contained within the internal wall of the vessel, the volume contained within the external wall of the vessel, or the like. In some embodiments, the volume refers to the volume of the whole vessel. In other embodiments, the volume refers to the volume of a predefined longitudinal section of the vessel or the volume of at least two predefined longitudinal sections of the vessel.

The diameter may correspond to the diameter of the lumen of the vessel, the distance between the centerline of the vessel and the internal wall of the vessel, the distance between the centerline of the vessel and the external wall of the vessel, etc. In some embodiments, a diameter refers to the diameter of the blood vessel at at least one predefined location along the length of the vessel. In this case, the diameter may refer to the diameter at a predefined location along the length of the blood vessel along a given radial direction. In another example, the diameter of the blood vessel may be determined for different radial directions and the diameter refers to the average diameter, the maximal diameter, the minimal diameter, etc.

In other embodiments, the diameter may be associated with a longitudinal section of the vessel. In this case, the diameter may refer to the average diameter of the longitudinal section of the vessel, the minimal diameter of the longitudinal section of the vessel, the maximal diameter of the longitudinal section of the vessel, etc.

The surface area may correspond to the surface area of the lumen of the vessel, the surface area of the internal wall of the vessel, the surface area of the external wall of the vessel, or the like. In some embodiments, the surface area refers to the surface area of the whole vessel. In other embodiments, the surface area refers to the surface area of a predefined longitudinal section of the vessel or the surface area of at least two predefined longitudinal sections of the vessel.

The length may correspond to a length along the lumen of the vessel (i.e., the length between two predefined points or landmarks on the lumen wall of the vessel), a length along the internal wall of the vessel (i.e., the length between two predefined points or landmarks on the internal wall of the vessel), a length along the external wall of the vessel (i.e., the length between two predefined points or landmarks on the external wall of the vessel), or the like. In some embodiments, the length refers to the length of the whole vessel. In other embodiments, the length refers to the length of a predefined longitudinal section of the vessel or the length of at least two predefined longitudinal sections of the vessel.

The asymmetry corresponds to the asymmetry of the change of one of the above-mentioned deformation parameters, e.g. the diameter, the volume, the surface area or the length, between the initial 3D model and the subsequent 3D model. In this case, the blood vessel is radially divided into a plurality of radial portions. For example, the blood vessel may be radially divided into eight 45 degrees octants. The value of the deformation parameter is determined for each radial portion for the initial 3D model the subsequent 3D model and the difference between the two values represent the change in the deformation parameter for each radial portion. The variation of the change in the deformation parameter shows the asymmetry of the deformation of the blood vessel. For example, the value of the deformation parameter may remain the same between the initial and subsequent 3D models for all of the radial portions except one, showing an asymmetry in the deformation of the blood vessel. In some embodiment, the blood vessel may further be longitudinally divided so that the blood vessel is divided into a plurality of subsections, each subsection extending longitudinally along a given distance along the length of the blood vessel and radially along a given radial length, and each positioned a respective longitudinal position and a respective radial position. A value of deformation parameter is then calculated for each subsection for the initial 3D model and the subsequent 3D model, and the difference between the two calculated values represents the change in the deformation parameter for each subsection of the blood vessel.

In at least some embodiments, the deformation determination procedure 308 further comprises a step of outputting information indicative of the determined deformation of the blood vessel, such as information about the growth of a blood vessel. For example, the information indicative of the determined deformation may be saved in memory. In the same or another example, the information indicative of the determined deformation may be provided for display. In this case, the information indicative of the determined deformation is sent to a display unit for display thereon.

In some embodiments, the step of determining a deformation of the blood vessel comprises determining a first deformation parameter value for the initial 3D model and a second deformation parameter value for the subsequent 3D model, i.e., a first value for a given deformation parameter for the initial 3D model and a second value for the same given deformation parameter for the subsequent 3D model. For example, the step of determining a deformation of the blood vessel may comprise determining a first diameter for the initial 3D model and a second diameter for the subsequent 3D model. In this case, it should be understood that the first and second deformation parameter values are determined for the same point (or landmark), section, portion or subsection on the initial 3D model and the subsequent 3D model.

In some embodiments, the deformation determination procedure 308 further comprises a step of comparing the determined deformation parameter to a predefined threshold. In some embodiments, the determined deformation parameter is compared to a maximum threshold and when the determined deformation parameter is equal to or greater than the maximum threshold, an alert is generated. The alert may be a visual alert, such as a written message, which may be provided for display.

In at least some embodiments, the step of determining a deformation of the blood vessel comprises determining a variation in at least one deformation parameter for the blood vessel. For example, the step of determining a deformation of the blood vessel comprises determining a variation in the diameter of the blood vessel or in at least one predefined section, portion or subsection of the blood vessel. In another example, the step of determining a deformation of the blood vessel comprises determining a variation in the diameter of the blood vessel or a variation in the diameter of at least one predefined section portion or subsection of the blood vessel. In a further example, the step of determining a deformation of the blood vessel comprises determining a variation in the surface area of the blood vessel or a variation in the surface area of at least one predefined section portion or subsection of the blood vessel. In still another example, the step of determining a deformation of the blood vessel comprises determining a variation in the length of the blood vessel or a variation in the length of at least one predefined section portion or subsection of the blood vessel.

In some embodiments, the deformation determination procedure 308 further comprises a step of comparing the determined variation in the deformation parameter to a predefined variation threshold. In some embodiments, the variation in the determined deformation parameter is compared to a maximum variation threshold and when the determined variation in the deformation parameter is equal to or greater than the maximum variation threshold, an alert is generated. The alert may be a visual alert, such as a written message, which may be provided for display.

In some embodiments, the deformation determination procedure 308 further comprises the steps of generating a graphical user interface (GUI) and providing the GUI for display. The GUI may comprise at least one determined deformation parameter and/or at least one determined variation in a deformation parameter. When a visual alert is generated, the GUI may comprise the visual alert.

In some embodiments, the GUI further comprises a visual representation of the initial 3D model and the subsequent 3D model, such as a visual representation the initial 3D model registered to the subsequent 3D model. In this case, at least one of the initial and subsequent 3D models may be see-through.

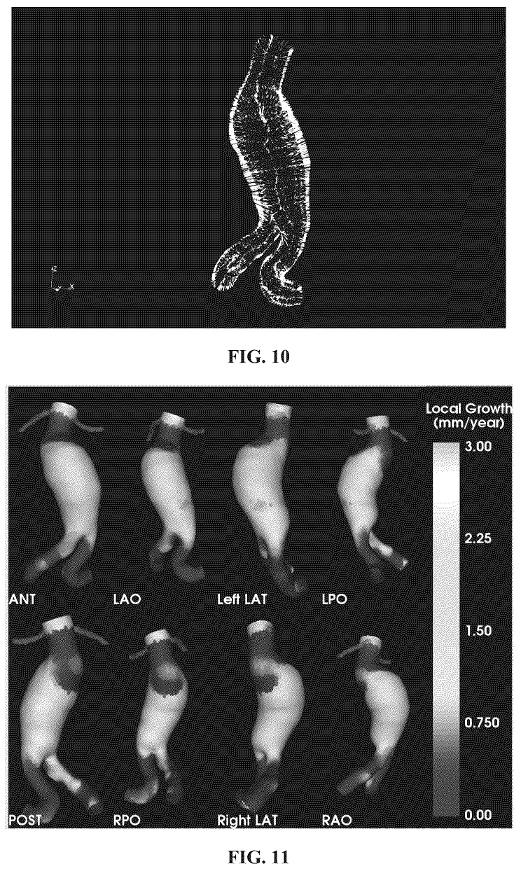

FIG. 10 illustrates a representation of a 3D model of a blood vessel which may be incorporated into the GUI. The illustrated representation is indicative of the variation of a given deformation parameter between an initial model and a subsequent model, the length of each white line being indicative the amplitude of the variation of the given deformation parameter.

In some embodiments, the GUI is interactive. In some embodiment, the GUI is designed as to allow the user to select, amongst a list of predefined deformation parameters, at least one deformation parameter for which he wants a value and/or a variation value. The list of predefined deformation parameters may comprise a diameter, a maximal diameter, a minimal diameter, an average diameter, a volume, a surface area, a length, an asymmetry, etc. In some embodiments, the GUI further allows the user to select point(s) and/or section, portion or subsection of the initial 3D model and/or the subsequent 3D model. In some embodiments, the GUI comprises predefined points (or landmarks) on the initial 3D model and/or the subsequent 3D model amongst which the user may select at least on point, and/or predefined sections, portions or subsections of the initial 3D model and/or the subsequent 3D model amongst which user may select at least one section, portion or subsection. In the same or other embodiments, the user may select at least one point and/or at least one section, portion or subsection directly on the displayed initial and/or subsequent 3D models using an input device such as a mouse. For example, the user may select a given point on the initial 3D model and further select to obtain the diameter at the selected point. In this case, the processor receives the identification of the selected deformation parameter, i.e., the diameter, and the selected point on the initial 3D model, determines the value of the diameter of the initial 3D model at the selected point and the value of the diameter of the subsequent 3D model at a point on the subsequent 3D model that corresponds to the selected point, and incorporates the determined diameters into the GUI for display to the user.

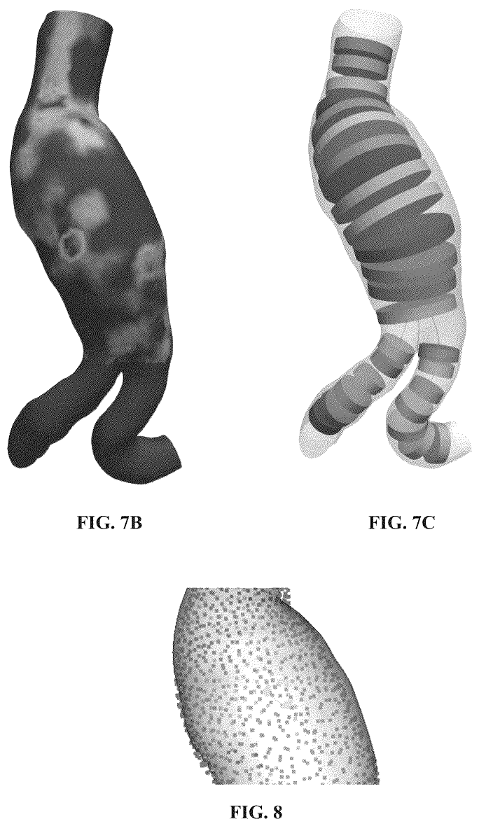

In some embodiments, the GUI comprises a representation of the blood vessel divided into a plurality of sections, portions or subsections and a respective color is assigned to each section, portion or subsection based on the respective variation in deformation parameter between the initial and subsequent 3D models determined for the section. For example, the average diameter may be determined for each section of the initial 3D model and the average diameter for each section of the subsequent 3D model (each section of the subsequent 3D model corresponding to a respective section of the initial 3D model). The variation in the average diameter is determined for each section based on the previously determined average diameters. A predefined number of ranges of possible values for the variation in average diameter is set and each range is assigned a respective color. Each section of the representation of the blood vessel is assigned the color of the range in which its determined variation in average diameter falls, as illustrated in FIG. 7A for example. It should be understood that instead of the average diameter of a section, other deformation parameters such as the volume or the surface area may be displayed to the user by assigning a color to each section of the representation of the blood vessel.

FIG. 11 illustrates eight different views of a 3D vessel model taken at different positions about the 3D model, the color of each point of the 3D model being indicative of a diameter growth in time, i.e. between an initial model acquired at a first point in time and a subsequent model acquired at a second point in time.

In some embodiments, a blood vessel growth can be described through measurements, such as the volume of the vessel, the surface area of the vessel (e.g., the surface area of the internal surface of the vessel or the surface area of the external surface of the vessel), the diameter or maximal diameter of the vessel and/or the length of the vessel, and/or these measurements applied to at least one predefined section of the vessel, e.g., the volume of at least a section of a vessel, the surface area of at least a section of a vessel (e.g., the surface area of the internal surface of at least a section of a vessel or the surface area of the external surface of at least a section of a vessel), the diameter or maximal diameter of at least a section of a vessel and/or the length of at least a section of a vessel. Along with sectional or global measurements, localized measurements such as the increase in surface area, increase in volumetric elements, or the local expansion of the vessel measured from predefined points in the model to the centerline of the model can be performed.

In some embodiments, measurements on the surface of the mesh of a model can be taken to obtain deformation of polygonal elements on the model, regional areas of deformation on the model, and/or volumetric measurements of growth on the model.

In some embodiments, measurements using the centerline can include diametric growth of the model and measurements of volumetric change in sections of the model.

In some embodiments, measurements can include mapping of data from one time point to another. Along with this, changes in the shape characteristics of the model and centerline can be obtained including growth in length of the model, asymmetry of the growth in the model. All measurements may be expressed as a function of time to show velocities and trajectories of the growth in the model.

In an embodiment in which a blood vessel growth is to be characterized, diameter, length and/or volume measurements are performed by the deformation determining procedure 308. The diameter taken at a same given point along the centerline of the 3D model is computer for both the initial 3D model and the subsequent 3D model. The difference between the diameter computed for the subsequent 3D model and that computed for the initial 3D model is indicative of an extension of the diameter of the blood vessel at the given point. The computed diameter may be the diameter of the lumen of the blood vessel, the diameter of the external wall of the blood vessel, and/or the like.

In one or more embodiments, the computed diameter corresponds to the maximal diameter within the cross-section taken at the given point along the centerline. In another embodiment, the computed diameter is the minimal diameter. In a further embodiment, the computed diameter is the average diameter.

In one or more embodiments, the deformation determining procedure comprises the calculation of volumes. The volume of a same section of the blood vessel is calculated for the initial 3D model and the subsequent 3D model. A growth of the blood vessel may be determined by comparing the volume computed for the subsequent 3D model to that computed for the initial 3D model. In one or more embodiments, the computed volume corresponds to the volume between two landmarks. In one or more embodiments, the computed volume corresponds to the volume of at least a section of the lumen of the blood vessel. In another embodiment, the computed volume corresponds to the volume of at least a section of the blood vessel.

In one or more embodiments, the deformation determining procedure comprises the calculation of lengths. The length between two landmarks or between two point of the 3D model is calculated for the initial 3D model and the subsequent 3D model. A deformation of the blood vessel may be determined by comparing the length computed for the subsequent 3D model to that computed for the initial 3D model.

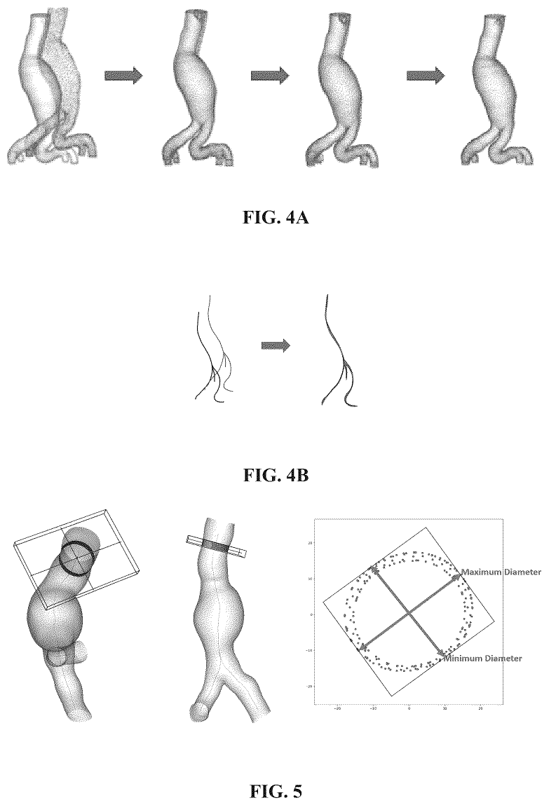

FIG. 5 illustrates a longitudinal section of a 3D model obtained using a common geometric space defined by the centerline of the vessel. The centerline of the vessel is used to compute and define normal and parallel directions in the vessel. The directions are then used to obtain the longitudinal sections of the vessel to understand asymmetric measures of the vessel.

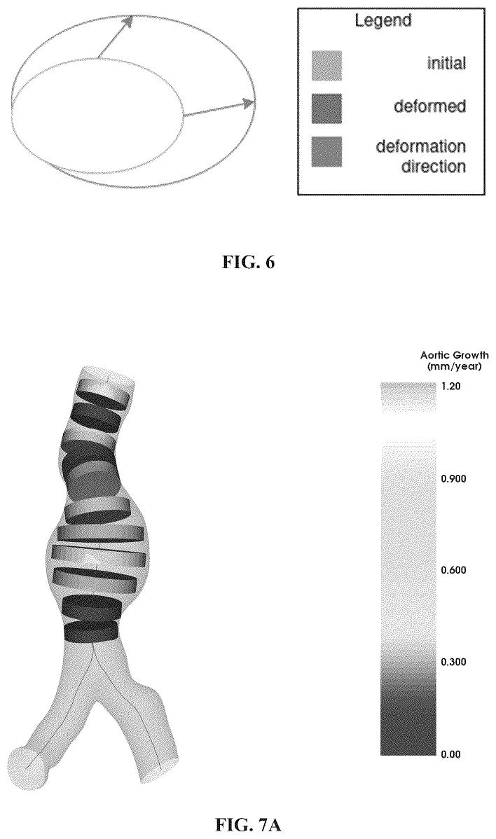

FIG. 6 shows the distribution of diameter measurements at a single level of the vessel geometry taken at a given point along the centerline. The diameter measurement is taken normal to the centerline of the vessel, where each data-point along the centerline produces a unique measurement at the given data-point for a given centerline. The measurement of the vessel may be taken from a common centerline that is aligned for both the initial and subsequent 3D model. In another example, the measurement of the vessel may be taken from the centerline of both the initial and subsequent 3D models. The measurement of deformation between the initial and subsequent 3D models using the sections is obtained through the previously described process. Furthermore, FIG. 6 shows the measures related to the direction and rotation of the deformation of the vessel. These measurements provide information on the evolution of tortuosity in the vessel during growth.

FIGS. 7A-7C show a distribution of the change in growth measurements generated for a vessel measured at a given set number of longitudinal sections that are oriented normal to the centerline of the vessel. At each longitudinal section, a section of the vessel geometry is taken from the full vessel geometric data set. The longitudinal section is then analyzed to determine changes in deformation parameter (or geometric measurement) in this section, such as changes in the maximum diameter, the minimum diameter, the equivalent circle diameter, the volume, and/or the like. The change in deformation parameter can then be computed by comparing the measurement for the initial 3D model and the subsequent 3D model. It should be understood that the number of longitudinal sections illustrated in FIGS. 7A-7C is exemplary only. In FIGS. 7A-7C, the color scale represents the rate of diametric growth where orange represents a higher rate of growth. in diameter, while blue represents little to no change.

FIG. 8 illustrates the measures of deformation or growth applied to multiple to the data-points on the model, connections, polygons, and regions of the model. Points on the model, are the points in 3-dimensional space that the model is composed of. Connections are the connectivity or edges of the polygons that are formed through connections between the points. Polygons refer to the shape created through the connections between points. Regions are defined as groups of polygons or points with a clear geometric definition, such as a volume, or a shape feature in the model, such as the iliac arteries or aneurysm body. The connections between data-points can be used to determine the deformation between a given data-point from one time-point to the next. The direction and magnitude of the deformation are obtained through the line that connects the two data-points. Connection information between data-points can be used to create geometric planes or polygons. The polygons that are defined by the connections between points in the data set are used to compute deformation with respect to shape characteristics of the polygons, such as areal deformations. The connections between polygons may be used to define volumes within the data set.

In one or more embodiments, for areal deformations, the change in area from initial 3D model to the subsequent 3D model is computed by using the bounded area for each polygonal element in the 3D model. The volumetric computations are performed through the bounded volume contained within multiple polygonal elements.

For deformations related to the change in the configuration of the surface of the model, of the points that compose the model, lines that connect those points, volumes that are bounded by elements can all be computed as deformations. The deformation is defined by identifying the equivalent data point for the initial 3D model and computing the new position of that point in the subsequent 3D model. These are used to compute the growth as a deformation between data points using the deformation gradient and continuum mechanic definitions of strain. The deformation gradient may be expressed as:

$${F}_{ij} = \frac{\partial {x}_{i}}{\partial {X}_{j}} = [ \begin{matrix}\frac{\partial {x}_{1}}{\partial {X}_{1}} & \frac{\partial {x}_{1}}{\partial {X}_{2}} & \frac{\partial {x}_{1}}{\partial {X}_{3}} \\ \frac{\partial {x}_{2}}{\partial {X}_{1}} & \frac{\partial {x}_{2}}{\partial {X}_{2}} & \frac{\partial {x}_{2}}{\partial {X}_{3}} \\ \frac{\partial {x}_{3}}{\partial {X}_{1}} & \frac{\partial {x}_{3}}{\partial {X}_{2}} & \frac{\partial {x}_{3}}{\partial {X}_{3}}\end{matrix} ] \qquad ( Eq . 1 )$$

where x is the subsequent configuration and X is the initial configuration.

From the deformation gradient, the left and right Cauchy-Green Strain tensors can be computed as:

$$C = {F}^{T} F \qquad ( Eq . 2 )$$

$$B = F {F}^{T} \qquad ( Eq . 3 )$$

where C is the right Cauchy-Green strain tensor, and B is the left Cauchy-Green strain tensor.

From the right Cauchy-Green strain tensor, the Green-Lagrange strain can be computed as:

$$E = \frac{1}{2} ( C - I ) \qquad ( Eq . 4 )$$

where E is the Green-Lagrange strain tensor.

The principal components of the Green-Lagrange strain tensor are obtained by computing the eigenvectors and eigenvalues for the tensor, as follows:

$$( E - λ I ) v = 0 \qquad ( Eq . 5 )$$

The eigenvalues are the principal values of strain, and the eigenvectors are the principal directions of strain. The maximum eigenvalue is taken as the maximum principal component of strain.

To obtain the volumetric deformation of a volumetric element in the mesh, the Jacobian is defined as:

$$J = det F \qquad ( Eq . 6 )$$

The Jacobian quantifies the change in volume for each volumetric elements in the model. The volumetric elements can be generated from a 3-dimensional surface mesh to determine the volume. The volumetric growth in a model can be used in the same way as the surface deformation described herein.

In some embodiments, each point of the 3D model may have information associated thereto. For example, functional data can include hemodynamic information, intraluminal thrombus information, strain information, obtained at time-point 1 at which the first medical image was acquired and mapped to time-point 2 at which the second medical image was acquired. This can be used to compute changes in function of the vessel.

It should be expressly understood that not all technical effects mentioned herein need to be enjoyed in each and every embodiment of the present technology. For example, embodiments of the present technology may be implemented without the user enjoying some of these technical effects, while other non-limiting embodiments may be implemented with the user enjoying other technical effects or none at all.

Some of these steps and signal sending/receiving are well known in the art and, as such, have been omitted in certain portions of this description for the sake of simplicity. The signals can be sent-received using optical means (such as a fiber-optic connection), electronic means (such as using wired or wireless connection), and mechanical means (such as pressure-based, temperature-based or any other suitable physical parameter based).

Modifications and improvements to the above-described implementations of the present technology may become apparent to those skilled in the art. The foregoing description is intended to be exemplary rather than limiting.

1. A method for determining a deformation in a blood vessel of a subject, the method being executed by a processor, the method comprising:
receiving an initial 3D model of the blood vessel of the subject, the initial 3D model having been generated from an initial medical image of the blood vessel acquired at an initial point in time;
receiving a subsequent 3D model of the blood vessel of the subject, the subsequent 3D model having been generated from a subsequent medical image of the blood vessel acquired at a subsequent point in time;
registering the initial 3D model to the subsequent 3D model, thereby obtaining registered 3D models;
determining at least one deformation parameter of the blood vessel based on the registered 3D models; and
outputting the at least one deformation parameter.

2. The method of claim 1, wherein said receiving the subsequent 3D model comprises:
receiving the subsequent medical image of the blood vessel;
segmenting the subsequent medical image, thereby obtaining a subsequent segmented image; and
generating the subsequent 3D model of the blood vessel based on the subsequent segmented image.

3. The method of claim 2, wherein said receiving the initial 3D model comprises:
receiving the initial medical image of the blood vessel;
segmenting the initial medical image, thereby obtaining an initial segmented image; and
generating the initial 3D model of the blood vessel based on the initial segmented image.

4. The method of claim 1, wherein the initial 3D model and the subsequent 3D model each comprise a 3D surface model.

5. (canceled)

6. The method of claim 1, wherein said registering the initial 3D model to the subsequent 3D model is performed using one of:
a rigid registration method and a deformable registration method; and
the rigid registration method, an affine registration method and the deformable registration method.

7. (canceled)

8. The method of claim 1, further comprising identifying landmarks in the 3D initial model and the 3D subsequent model.

9. The method of claim 8, wherein said registering the initial 3D model to the subsequent 3D model is performed based on the identified landmarks.

10. (canceled)

11. The method of claim 1, wherein said registering the initial 3D model to the subsequent 3D model comprises:
determining a first centerline of the initial 3D model;
determining a second centerline of the subsequent 3D model; and
registering the first centerline to the second centerline.

12. The method of claim 1, wherein said determining at least one deformation comprises one of:
determining a deformation gradient based on the registered 3D models; and
determining a given deformation parameter for the initial 3D model and determining the given deformation parameter for the subsequent 3D model.

13. (canceled)

14. The method of claim 12, wherein the given deformation parameter comprises at least one of a diameter, a volume, a length, a surface area, an equivalent circle diameter and an asymmetry.

15. A system for determining a deformation in a blood vessel of a subject, the system comprising:
a processor; and
a non-transitory storage medium operatively connected to the processor, the non-transitory storage medium comprising computer-readable instructions stored thereon;
the processor, upon executing the computer-readable instructions, being configured for:
receiving an initial 3D model of the blood vessel of the subject, the initial 3D model having been generated from an initial medical image of the blood vessel acquired at an initial point in time;
receiving a subsequent 3D model of the blood vessel of the subject, the subsequent 3D model having been generated from a subsequent medical image of the blood vessel acquired at a subsequent point in time;
registering the initial 3D model to the subsequent 3D model, thereby obtaining registered 3D models;
determining at least one deformation of the blood vessel based on the registered 3D models; and
outputting the at least one deformation parameter.

16. The system of claim 15, wherein the processor is further configured for:
receiving the subsequent medical image of the blood vessel;
segmenting the subsequent medical image, thereby obtaining a subsequent segmented image; and
generating the subsequent 3D model of the blood vessel based on the subsequent segmented image.

17. The system of claim 16, wherein the processor is further configured for:
receiving the initial medical image of the blood vessel;
segmenting the initial medical image, thereby obtaining an initial segmented image; and
generating the initial 3D model of the blood vessel based on the initial segmented image.

18. The system of claim 15, wherein the initial 3D model and the subsequent 3D model each comprise a 3D surface model.

19. (canceled)

20. The system of claim 15, wherein the processor is configured for said registering the initial 3D model to the subsequent 3D model using one of:
a rigid registration method and a deformable registration method; and
the rigid registration method, an affine registration method and the deformable registration method.

21. (canceled)

22. The system of claim 15, wherein the processor is further configured for identifying landmarks in the 3D initial model and the 3D subsequent model.

23. The system of claim 22, wherein the processor is configured for said registering the initial 3D model to the subsequent 3D model based on the identified landmarks.

24. (canceled)

25. The system of claim 15, wherein said registering the initial 3D model to the subsequent 3D model comprises:
determining a first centerline of the initial 3D model;
determining a second centerline of the subsequent 3D model; and
registering the first centerline to the second centerline.

26. The system of claim 15, wherein the processor is configured for determining one of:
a deformation gradient based on the registered 3D models; and
a given deformation parameter for the initial 3D model and the given deformation parameter for the subsequent 3D model.

27. (canceled)

28. The system of claim 26, wherein the given deformation parameter comprises at least one of a diameter, a volume, a length, a surface area, an equivalent circle diameter and an asymmetry.
